(19) United States
(12) Patent Application Publication
(10) Pub. No.: US 2004/0019258 A1
(43) Pub. Date: Jan. 29, 2004

(54) Detecting removal of a medical device from a station

(76) Inventors: Gregory T. Kavounas, Kirkland, WA (US); Randy L. Merry, Woodinville, WA (US); Shawn R. Bertagnole, Lake Stevens, WA (US)

Correspondence Address: SHUMAKER & SIEFFERT, P. A., 8425 SEASONS PARKWAY, SUITE 105, ST. PAUL, MN, 55125, US

(21) Appl. No.: 10/357,301

(22) Filed: Jan. 31, 2003

Related U.S. Application Data
(60) Provisional application No. 60/394,981, filed on Jul. 9, 2002.

(51) Int. Cl. A61B 5/00

(52) U.S. Cl. 600/300; 600/594

(57) ABSTRACT

Techniques are described for detecting removal of a medical device from a station and activating an alarm in response to the detected removal. More specifically, the station includes a detector that detects removal of the medical device and causes the alarm to activate upon detecting the removal. For example, the station may include an optical detector that receives an optical signal from a light transmitter on the medical device. When the optical detector does not receive a signal from the medical device, the optical detector sends a signal to activate the alarm. The optical detector may, for example, not receive an optical signal when the medical device is too far from the optical detector or oriented in an improper direction. The sensitivity of the detector may further be calibrated to allow different detection ranges to be defined for the station.

This application claims priority from U.S. Provisional Application Serial No. 60/394,981, filed Jul. 9, 2002, the entire content of which is incorporated herein by reference.

### TECHNICAL FIELD

The invention relates to medical devices, and more particularly, to detecting removal of a medical device from a station.

### BACKGROUND

Cardiac arrest is a life-threatening medical condition that may be treated with external defibrillation. External defibrillation includes applying electrodes to a chest of a patient and delivering an electric shock to the patient to depolarize a heart of the patient and restore normal sinus rhythm. The chances that the heart of the patient can be successfully defibrillated increase significantly if a defibrillation pulse is applied quickly.

In some cases, the patient's need is urgent and the patient cannot wait for trained personnel, such as paramedics, emergency medical technicians, or others trained in defibrillation techniques, to arrive. In recognition of the need for prompt treatment and the advantages of early defibrillation, automated external defibrillators (AEDs) are becoming more commonplace, and are available in venues such as airports, health clubs and auditoriums.

Within the venues described above, an AED may be housed within a storage unit. The storage unit may protect the AED from the surrounding environment. For example, the storage unit may protect the AED from being disturbed by passersby. Further, the storage unit may protect the AED from theft and tampering. The storage unit, for example, may have a lock that must be unlocked before opening the storage unit. The storage unit may further include an alarm that is activated upon opening of a door of the storage unit. However, if a portion of the door of the storage unit is designed for breaking, such as a storage unit for a fire extinguisher, the door will remain shut and the alarm will not sound upon removing the AED. Further, the alarm may falsely sound during routine maintenance, for instance, during a routine test of the AED.

### SUMMARY

In general, the invention is directed to techniques for detecting removal of a medical device from a station and activating an alarm in response to the detected removal. More specifically, a station may include a detector that detects removal of a medical device from the station. Upon detecting removal of the medical device, the detector causes the alarm to activate in order to provide notification an emergency situation or unauthorized removal of the medical device.

The notification may take the form of an audible or visible alarm for notification of people in the vicinity of the station. For example, the alarm, when activated, may sound a buzzer, a siren or any other audible alarm. Further, the activated alarm may emit a strobe light or other visible alarm. The alarm may be deactivated after a defined period of time or via an alarm deactivation. The alarm deactivation may be used to deactivate and reset a sounding alarm or deactivate the alarm before it sounds. The alarm deactivation may, for example, be used to deactivate the alarm in order to perform routine maintenance to the medical device.

Alternatively, the notification may involve activation of a remote alarm or communication with a remote safety agency. For example, the station may directly contact a safety agency, such as an alarm monitoring service or an emergency services agency, in response to the removal of the medical device from the station. For instance, the station may send an advisory message to the safety agency alerting the safety agency to the emergency situation currently in progress. The advisory may include location information as well as any other pertinent information.

In one embodiment, the invention provides a system comprising a station to support an emergency medical device, a detector to detect removal of the medical device from the station, and an alarm that is activated in response to the detected removal of the medical device.

In another embodiment, the invention provides a method comprising detecting removal of an emergency medical device from a station that supports the emergency medical device and activating an alarm responsive to the removal of the medical device from the station.

In another embodiment, the invention provides a system comprising means for supporting a medical device, means for detecting removal of the medical device from the station, and means for alerting people in the vicinity that the medical device has been removed.

The invention can provide a number of advantages. In general, the invention provides techniques for early and accurate detection of removal of a medical device from a station. The techniques of the invention may be used to detect removal of the medical device from a storage unit without needing the storage unit to have a door, which further needs to be opened for detection. Further, routine maintenance may be performed without falsely activating the alarm. For example, depending on the detector used, the medical device may be moved within a detection range without sounding the alarm. In this manner, routine maintenance such as battery replacement or calibration, may be performed without activating the alarm.

The techniques of the invention allow the detector to be calibrated, for example, during installation. The ability to calibrate the detector allows different detection ranges to be defined for the station. For example, a sensitivity of the detector may be calibrated in order to increase the distance by which the medical device may be moved before removal is detected.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating a system in which an alarm is activated in response to detecting removal of a medical device from a station according to the present invention.

FIG. 2 is a block diagram illustrating an automated external defibrillator (AED) removal detection system in which a switch detects removal of an AED from a station and activates an alarm in response to the removal of the AED.

FIG. 3 is a block diagram illustrating another AED removal detection system in which a suspended switch is used to detect removal of an AED from a station.

FIG. 4 is a block diagram illustrating another AED removal detection system that uses a receptacle plug sensor to detect removal of an AED from a station.

FIG. 5 is a block diagram illustrating another AED removal detection system that uses an optical detector to detect removal of an AED from a station.

FIG. 6 is a block diagram illustrating another AED removal detection system that uses a wireless receiver to detect removal of an AED from a station.

FIG. 7 is a block diagram of an exemplary storage unit that activates an alarm upon detecting removal of a medical device.

FIG. 8 is a block diagram illustrating a side view of another exemplary storage unit.

FIG. 9 is a block diagram illustrating a system that provides direct communication with a safety agency as well as activation of an alarm in response to detecting removal of a medical device from a station.

FIG. 10 is a block diagram illustrating an exemplary embodiment of communications circuitry within a station for automatic initiation of direct contact between station and a safety agency.

FIG. 11 is a flow diagram illustrating installation of a station and calibration of a detector within the station for detecting removal of a medical device from the station.

FIG. 12 is a block diagram illustrating operation of a removal detection system upon detecting removal of a medical device from a station.

### DETAILED DESCRIPTION

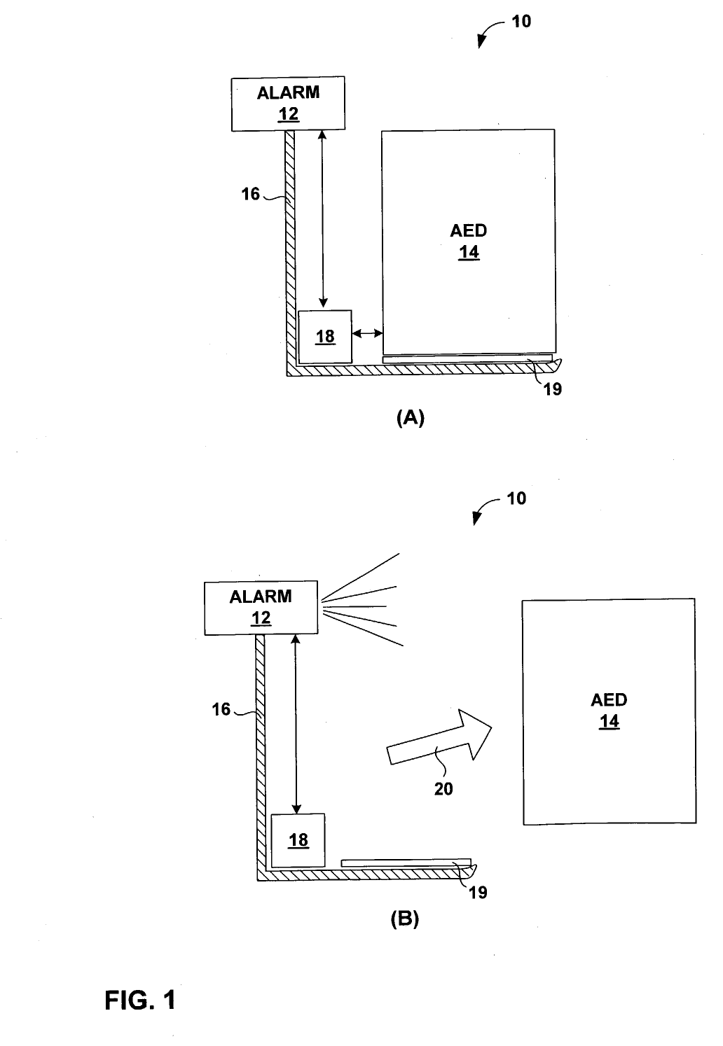

FIG. 1 is a block diagram illustrating a system 10 in which an alarm 12 is activated in response to detecting removal of a medical device, such as an automated external defibrillator (AED) 14, from a station 16. More specifically, a detector 18 detects removal of AED 14 from station 16 and causes alarm 12 to activate upon detecting the removal of AED 14 from station 16.

FIG. 1A illustrates AED 14 in a “mounted” state. The term “mounted” state refers to a state in which AED 14 is within, on, or otherwise supported by station 16. The term “mounted” may further refer to AED 14 being in close proximity to station 16. For example, AED 14 may be in the mounted state when AED 14 is within a detectable range of a detector associated with station 16. FIG. 1B illustrates AED 14 being removed according to arrow 20 to an unmounted or removed state.

In the example of FIG. 1A, a lower portion of station 16 supports AED 14. More specifically, AED 14 rests on station 16 much like a shelf. Station 16 may further include a non-skid surface 19 that prevents AED 14 from inadvertently sliding off of station 16. Nonskid surface 19 may, for example, be formed by applying a patch of coarse material to the surface of station 16. Alternatively, station 16 may support AED 14 via a hook, a mount, or other supporting means. Further, in some cases, station 16 may be a storage unit such as a wallbox or cabinet that encloses AED 14 and protects it from the surrounding environment. In either case, station 16 may be mounted on a wall, recessed within a wall, or be free standing.

Station 16 includes a detector 18 to detect whether AED 14 remains in a mounted state or is removed from station 16. Detector 18 may be implemented via various proximity sensors, such as a mechanical switch that is activated upon removal of AED 14 from the station 16, a capacitive sensor that senses a change in capacitance upon removal of AED 14 from the station 16, an optical emitter-detector circuit, a wireless sensor that detects removal of AED 14 from station 16 when the wireless sensor no longer receives a signal, an optical sensor that detects removal of AED 14 from station 16 when the optical sensor no longer receives an optical signal, and a receptacle plug sensor that detects removal of AED 14 from station 16 when the receptacle plug is unplugged.

Upon detector 18 detecting removal of AED 14 from station 16, detector 18 causes alarm 12 to activate. Alarm 12 may, for example, be coupled to detector 18 via a wireless connection or a wired connection. Alarm 12 may be a visual alarm such as a strobe light, an audible alarm such as a siren or a buzzer, or a combination of visual and audible alarms. FIG. 1B illustrates the activation of alarm 12 in response to removal of AED 14, indicated by arrow 20.

AED 14 may, for example, be out of a detectable range of a wireless sensor forming part of detector 18. Although in the example of FIG. 1, alarm 12 and detector 18 reside within station 16, alarm 12 and detector 18 may reside within AED 14. In this manner, detector 18 may detect, for example, the presence of station 16. Upon removal of AED 14 from station 16, the alarm within AED 14 activates to alert people in the vicinity of the station 16 of an emergency situation or unauthorized removal of AED 14.

Further, station 16 may be configured to contact a safety agency upon removal of AED 14. Contacting a safety agency upon removal of AED 14 from station 16 may involve, for example, sending an advisory from station 16 to the safety agency using a telecommunication link. Alternatively, AED 14 may include a communication interface to send the advisory the safety agency. The safety agency may be, for example, an emergency services agency operating an Emergency Medical System such as 9-1-1 in the United States, or a security monitoring agency.

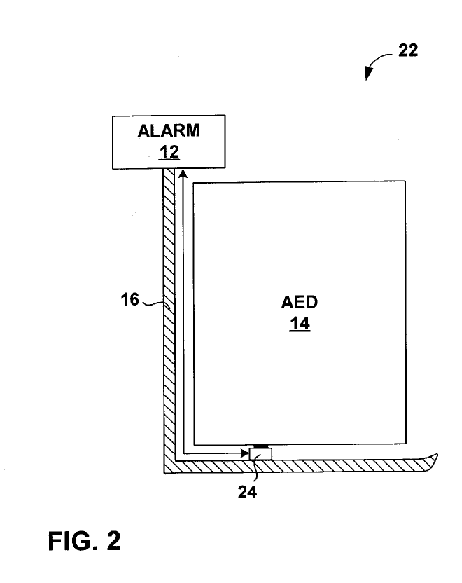

FIGS. 2-7 illustrate a variety of AED removal detection systems that actuate an alarm in response to removal of AED 14 from station 16. FIG. 2 is a block diagram illustrating an AED removal detection system 22 in which a switch 24 detects removal of an AED 14 from a station 16. AED 14 may rest on switch 24 and the weight of AED 14 may serve to depress an actuator associated with switch 24. Removing AED 14 from station 16 activates switch 24 by no longer depressing switch 24 and, in turn, causing alarm 12 to activate.

As shown in the example of FIG. 2, switch 24 resides on a bottom portion of station 16. In this manner, AED 14 may rest on top of switch 24. Alternatively, switch 24 may reside on a back plane of station 16. In this configuration, AED 14 may rest on the bottom portion of station 16 and lean against the back plane of station 16, depressing switch 24. Switch 24 may be a micro switch, a pin switch, a plate switch, or any other mechanical or electrical switch.

Switch 24 may be coupled to alarm 12 either via a wired connection or a wireless connection. Upon removal of AED 14, switch 24 opens or closes, depending on the normal state of the switch, and sends a signal to alarm 12 in response to switch movement. Alarm 12 activates in response to the signal from switch 24 to alert people within a close vicinity of an emergency situation or unauthorized removal of AED 14. Further, station 16 may contact a safety agency upon removal of AED 14, as discussed above.

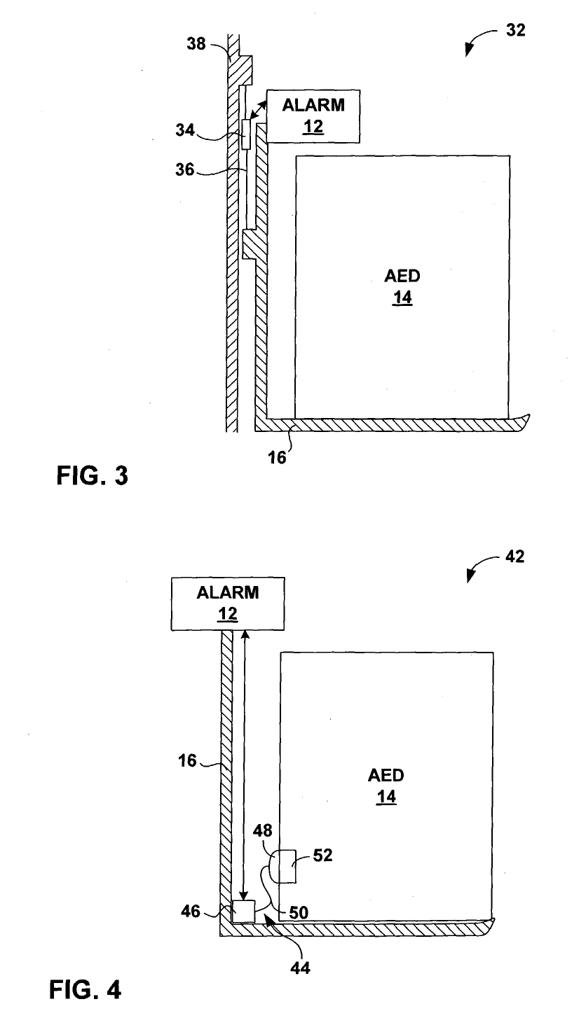

FIG. 3 is a block diagram illustrating an AED removal detection system 32 in which a suspended switch 34 detects removal of an AED 14 from a station 16. Removing AED 14 from station 16 activates switch 34, in turn causing alarm 12 to activate.

As shown in the example of FIG. 3, station 16 suspends from a wall 38. More particularly, a suspension support 36 and suspended switch 34 interconnect a protruding portion of wall 38 to station 16. Station 16 may also include a protruding portion to which an end of suspension support 36 connects. In this manner, the protruding sections of wall 38 and station 16 may connect to opposite ends of suspension support 36. Alternatively, suspension support 36 may connect to other parts of station 16. For example, suspension support 36 may connect to a top portion of station 16.

Wall 38 may further include a track (not shown) that guides station 16 to prevent swaying or other unnecessary side-to-side movement of station 16. For example, the protruding portion of station 16 may insert into the track to prevent unwanted side-to-side movement.

Suspension support 36 may include a cable, a wire or the like that has a tensile strength large enough to hold the weight of station 16 and AED 14. Suspended switch 34 activates when the weight of station 16 and AED 14 changes. More particularly, upon removal of AED 14 from station 16, suspended switch 34 detects a change in weight and activates in response to the change in weight. For example, suspended switch 34 may activate when the supported weight falls below a threshold weight. Suspended switch 34 sends a signal to alarm 12, in turn activating alarm 12 to notify people in the vicinity of an emergency situation in progress or unauthorized removal of AED 14. As with system 22 of FIG. 2, station 16 may further contact a safety agency upon removal of AED 14.

FIG. 4 is a block diagram illustrating an AED removal detection system 42 in which a receptacle plug detector 44 detects removal of an AED 14 from a station 16. Receptacle plug detector 44 includes a sensor 46, a plug 48, and a cord 50. Plug 48 inserts into a port 52 of AED 14. Alarm 12 activates upon removal of plug 48 from port 52.

More specifically, as AED 14 is removed from station 16, cord 50 extends until cord 50 reaches a maximum length. Upon reaching the maximum length and being pulled further, plug 48 ejects from port 52. Sensor 46 detects ejection of plug 48 and sends a signal to activate alarm 12. Sensor 46 may detect ejection of the plug via failure to receive an electrical signal across terminals in the plug that were electrically coupled by terminals in the port. For example, while plug 48 is inserted in port 52 sensor 44 receives an electrical signal from AED 14. Removal of AED 14 causes sensor 44 to no longer receive the electrical signal. In response to the loss of the electrical signal, sensor 44 issues a signal to activate alarm 12.

Although in the example of FIG. 4 the receptacle plug detector 44 is attached to station 16, receptacle plug detector 44 may be attached to AED 14. In this manner, sensor 46, plug 48, and cord 50 extend from AED 14 and couple to a port within station 16.

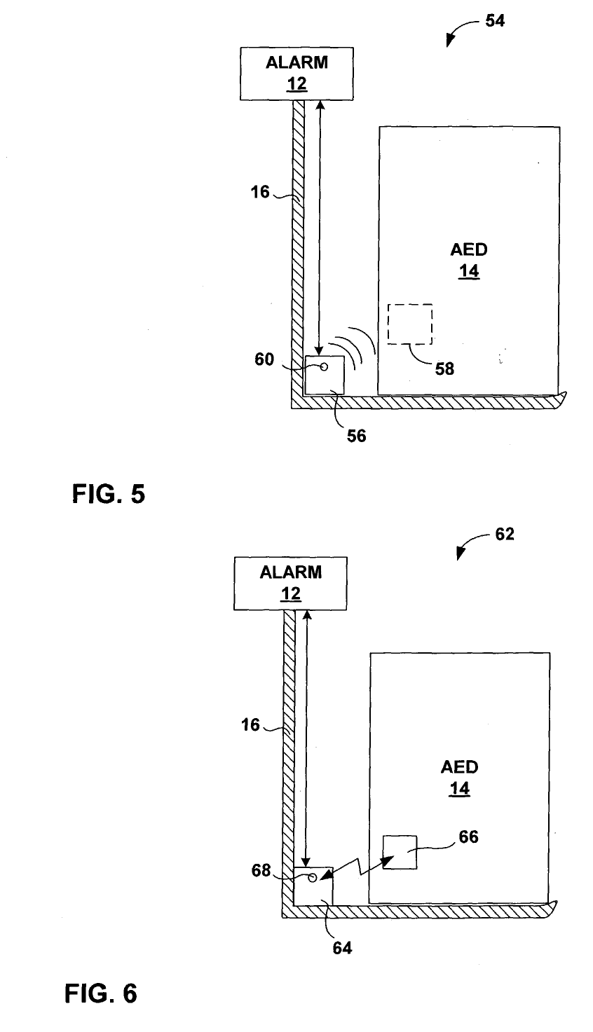

FIG. 5 is a block diagram illustrating an AED removal detection system 54 in which an optical detector 56 detects removal of an AED 14 from a station 16. Removing AED 14 from station 16 activates optical detector 56, in turn causing alarm 12 to activate.

Station 16 includes an optical detector 56 that receives an optical signal from AED 14. The optical signal serves as assurance that AED 14 is in the present state. AED 14 may include a light transmitter 58 that emits an optical signal to optical detector 56. The optical signal may be in the visible portion, the infrared (IR) portion, or other portions of the light spectrum. The optical signal transmitted by light transmitter 58 may further be characteristic of the respective AED 14 that emits the signal. The optical signal transmitted by light transmitter 58 may include, for example, a serial number of AED 14 or other information associated AED 14.

When optical detector 56 does not receive a signal from AED 14, optical detector 56 sends a signal to activate alarm 12. Optical detector 56 may, for example, not receive an optical signal when AED 14 is too far from optical detector 56 or oriented in an improper direction. In these cases, optical detector 56 fails to receive an expected optical signal.

Optical detector 56 may further include a control 60 to adjust the sensitivity of optical detector 56. Optical detector 56 may be adjusted to increase or decrease the strength of the optical signal that optical detector 56 must receive in order to consider AED 14 to be in the present state. In this manner, optical detector 56 may be calibrated to sense AED 14 at different distances. Calibrating optical detector 56 may adjust the detection range of station 12. Calibration using control 60 may occur, for example, during installation of station 16.

However, AED 14 may not transmit an optical signal. Instead, station 16 may include a transceiver. The transceiver may emit a querying optical signal to AED 14 and wait for a signal to be returned. A surface of AED 14 may, for example, be optically passive and reflect the optical signal back to the transceiver. The surface of AED 14 may be specially designed to have good reflection characteristics.

FIG. 6 is a block diagram illustrating an AED removal detection system 62 in which a wireless receiver 64 detects removal of an AED 14 from a station 16. Removing AED 14 from station 16 activates wireless receiver 64, in turn causing alarm 12 to activate.

System 62 operates in the same manner as system 54 of FIG. 5. Specifically, station 16 includes a wireless receiver 54 that receives a wireless signal from a wireless transmitter 66 in AED 14. The wireless signal transmitted by wireless transmitter 66 may be in the radio frequency (RF) portion, microwave portion, or other portions of the light spectrum.

When wireless detector 64 does not receive a signal, i.e., AED 14 is out of range or oriented improperly, wireless detector 64 sends a signal to activate alarm 12. Wireless detector 64 may further include a knob 66 to adjust the sensitivity of wireless detector 64. Wireless detector 64 may be adjusted to increase or decrease the strength of the wireless signal that wireless detector 64 must receive in order to consider AED 14 to be in the present state. In this manner, wireless detector 64 may be calibrated to sense AED 14 at different distances.

Alternatively, AED 14 may not transmit a wireless signal. Instead, station 16 may include a transceiver that emits a querying wireless signal to AED 14 and waits for a signal to be returned. AED 14 may include a passive element that retransmits or reflects the querying wireless signal back to the transceiver of station 16.

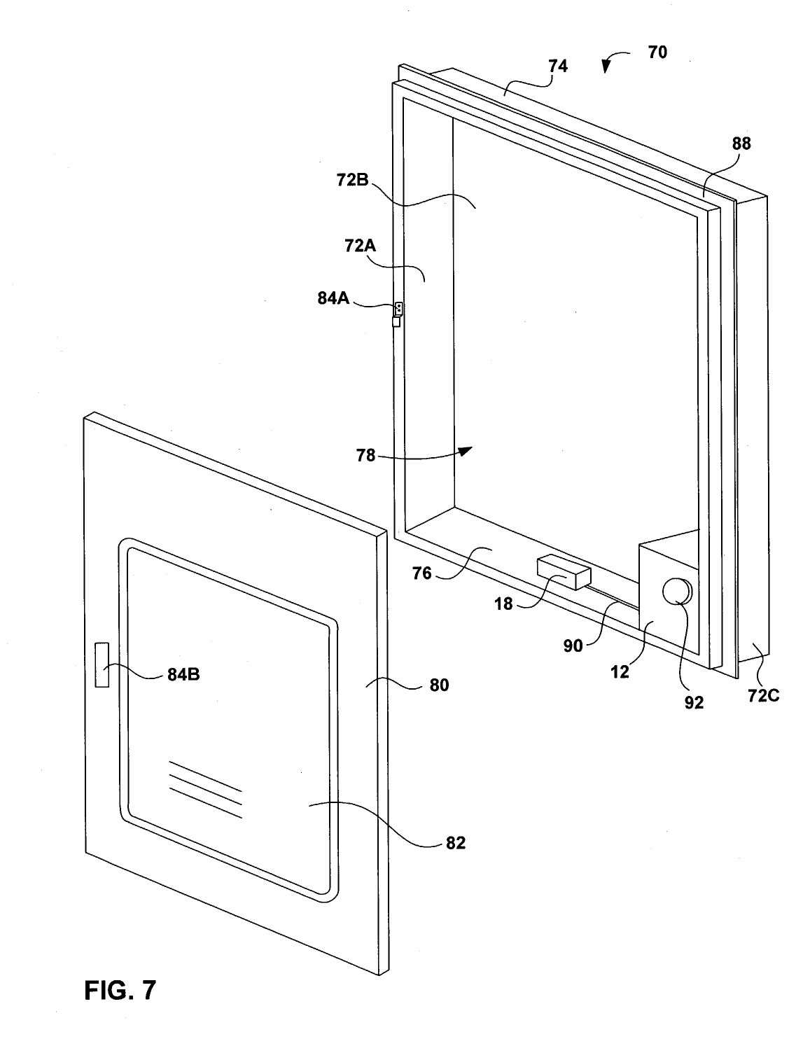

FIG. 7 is a block diagram of an exemplary storage unit 70 that activates an alarm 12 upon detecting removal of a medical device, such as AED 14 of FIG. 1. Storage unit 70 comprises a plurality of wall sections 72A-72C (“72”), a top section 74, and a bottom section 76 that define an interior compartment 78 of sufficient size to house a medical device. Storage unit 70 may further include a door section 80 to enclose the medical device to protect the medical device from the surrounding environment. Door section 80 may include a translucent section 82 to allow visibility into interior compartment 78. In this manner, the contents of storage unit 70 may be visible to an outside observer when door section 80 is closed. Translucent section 82 may be constructed of a translucent material such as a synthetic plastic, glass, or the like. Storage unit 70, including wall sections 72, top section 74, bottom section 76, and door section 80 (not including translucent section 82), may be made from steel or other rigid, lightweight material.

Storage unit 70 includes locking mechanisms 84A-84B (“84”) to secure door section 80 in a closed position. Locking mechanism 84A is mounted on a wall section 72 of storage unit 70 and locking mechanism 84B is mounted on door section 80. When door section 80 is closed, locking mechanism 84B receives locking mechanism 84A and secures door section 80 in the closed position. In order to open door 80, locking mechanism 84B may be pushed upward and door 80 may be pulled open.

Storage unit 70 is typically mounted onto a surface of a wall. Storage unit 70 may, for example, be mounted on a wall in venues such as airports, health clubs and auditoriums. In one embodiment of the invention, storage unit 70 may include a retaining flange 88 that extends around storage unit 70 to allow storage unit 70 to be at least partially recessed into the wall. Recessing storage unit 70 into the wall helps to minimize the amount of space required to accommodate storage unit 70. However, in venues where the walls are made of concrete or brick, storage unit 70 may protrude from the wall. Alternatively, storage unit 70 may attach to a base to allow the storage unit 70 to be free standing.

Storage unit 70 further includes a detector 18 that detects the presence of the medical device. Detector 18 is coupled to an alarm 12 via a wire 90. Alternatively, detector 18 may be coupled to alarm 12 via a wireless coupling or other coupling means. Upon detecting removal of the medical device from storage unit 70, a signal is sent via wire 90 to activate alarm 12. Alarm 12 is activated to alert people in the vicinity of an emergency situation in progress or unauthorized removal of the medical device. Detector 18 may activate alarm 12 whether door section 80 of storage unit 70 is opened or remains closed. Alarm 12 may, for example, still activate when translucent section 82 is broken to remove the medical device stored in storage unit 70.

An alarm deactivation 92 may be connected in circuit to alarm 12. Alarm deactivation 92 may reset the alarm system of storage unit 70 after activation. Alarm deactivation 92 may further totally deactivate alarm 12. Alarm 12 may, for example, be totally deactivated in order to perform routine maintenance to the medical device stored in storage unit 70. Alarm deactivation 92 may include a button, a switch, a dial or other input medium. Alarm deactivation 92 may, for example, be a controlled by a key switch. In this manner, only authorized personnel may deactivate alarm 12. Alternatively, alarm deactivation 92 may be an alarm deactivation timer. The deactivation timer may be initiated when alarm 12 is activated. Upon expiration of the deactivation timer, alarm 12 is deactivated and reset.

Detector 18 may be implemented via various proximity sensors including a mechanical switch, a capacitive sensor, an emitter-detector circuit, a wireless detector, an optical detector, a receptacle plug sensor, or similar proximity sensor. Alarm 12 may be a visual alarm such as a strobe light, an audible alarm such as a siren or a buzzer, or a combination visual and audible alarm. Although alarm 12 of FIG. 7 is illustrated within storage unit 70, alarm 12 may reside on an outside portion of storage unit 70, on a wall to which storage unit 70 is mounted, or the like. For example, alarm 12 may reside on door section 80 of storage unit 70.

The medical device stored in storage unit 70 may be supported by bottom section 76. However, the medical device may be stored within storage unit 70 via any storage configuration. For example, the medical device may be suspended from top portion 74 of storage unit 70, similar to system 32 of FIG. 3. Further, storage unit may include a mount to support the medical device. The mount may include, for example, a bracket connected to wall 72B of storage unit 70 that supports the medical device.

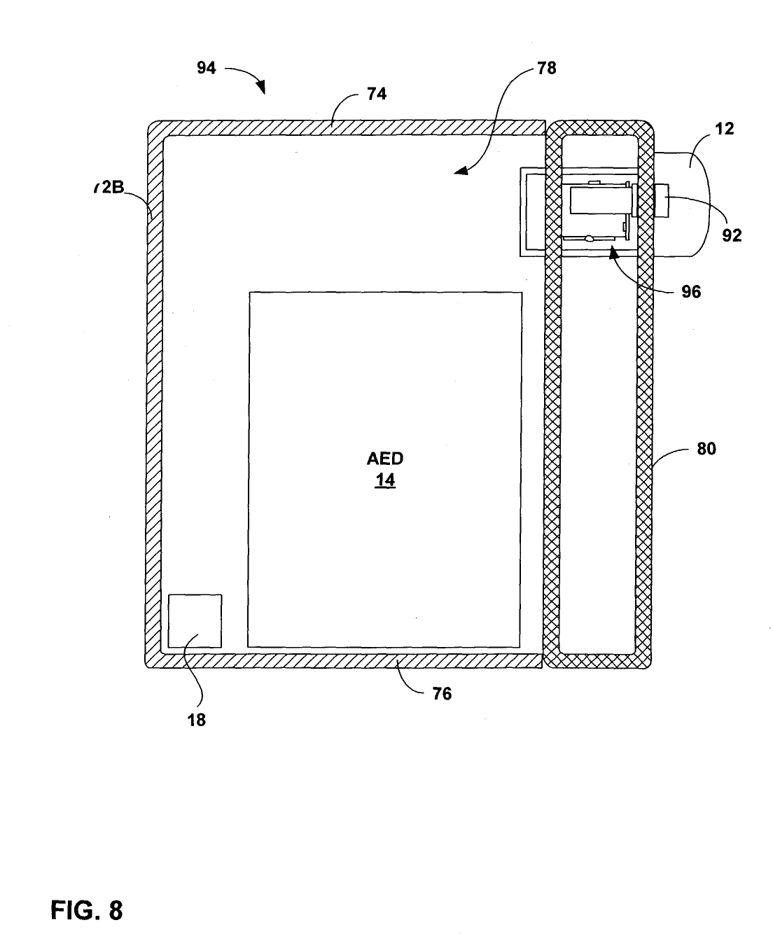

FIG. 8 is a block diagram illustrating a side view of another exemplary storage unit 95. In the example of FIG. 8, an AED 14 rests within storage unit 95 and, more particularly, within interior compartment 78. As described above, interior compartment 78 is formed via walls 72 (of which wall 72B is shown), top section 74, bottom section 76, and door 80. Interior compartment 78 may be formed to fit a small or large AED. Further interior compartment may be designed to hold AED 14 along with additional items. For instance, interior compartment 78 may further hold a first aid kit and any other medical or non-medical items.

A detector 18 detects when AED 14 is removed from storage unit 78. Detector 18 causes alarm 12 to sound in response to removal of AED 14. In the example illustrated in FIG. 8, alarm 12 resides on the outside of door 80. As described above, however, alarm 12 may reside anywhere within or on storage unit 95. Alarm deactivation 92 also resides on the outside of door 80. Alarm deactivation 92 is connected to alarm 12 via circuit 96. Circuit 96 allows alarm 12 to be deactivated and reset after sounding in response to removal of AED 14.

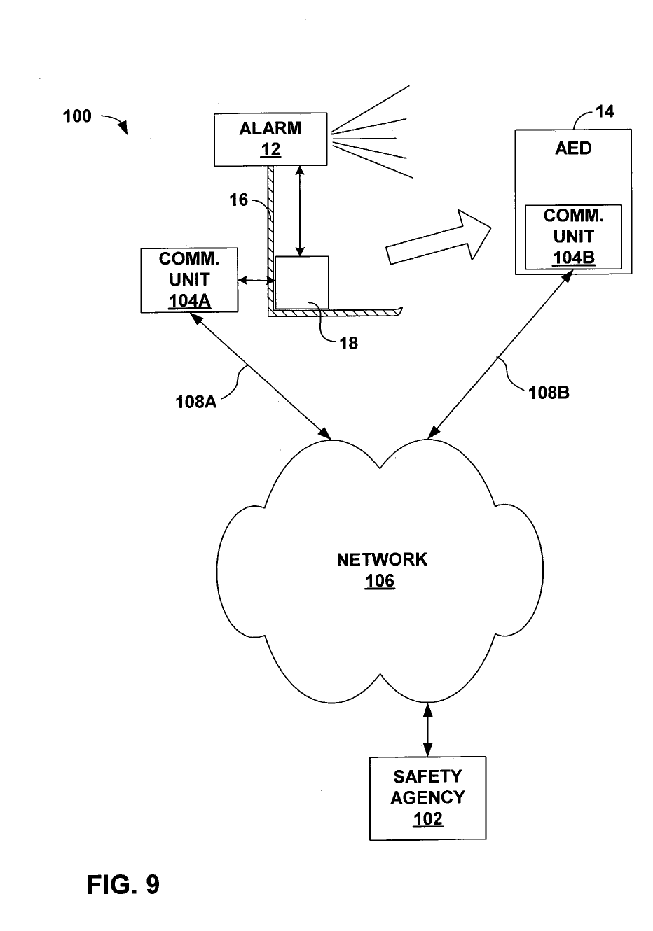

FIG. 9 is a block diagram illustrating a system 100 that provides direct communication with a safety agency 102 as well as activation of an alarm 12 in response to detecting removal of a medical device, such as AED 14. Direct communication between AED 14 and safety agency 102 may be initiated automatically upon removing AED 14 from station 16, prompting early notification and arrival of emergency personnel. Safety agency 102 may be, for example, an Emergency Medical System such as 9-1-1 in the United States, or a security monitoring agency.

As shown in FIG. 9, direct communication with safety agency 102 may be initiated by station 16 or by AED 14 via communication units 104A and 104B (“104”), respectively. Communication units 104 are coupled to a network 106 via links 108A and 108B (“108”), respectively. More than one link 108 may couple communication units 104 to network 106 in order to provide alternative communication paths between safety agency 14 and station 16 or AED 14. Communication units 104 may include a network card, a wireless local area network (WLAN) card, a mobile phone, an infrared (IR) card, a modem, or any combination thereof. Communication units 104 may instead couple station 16 or AED 14 and a communication device that is already coupled to network 18. For example, communication unit 104A may electrically couple station 16 to a mobile phone via a connector that connects to the mobile phone and station 16.

Network 106 may be a combination of network architectures, including a public switched telephone network (PSTN), an integrated services digital network (ISDN), an Internet protocol (IP) network, a local area network (LAN), a wide area network (WAN), a wireless communications network, or an asynchronous transfer mode (ATM) network. Links 108 may be wireless links, wired links, optical links or the like.

Detector 18 of station 16 detects removal of AED 14 from station 16. Upon detecting removal of AED 14 from station 16, detector 18 causes alarm 12 to activate in order to notify people in the vicinity of an emergency situation in progress or an unauthorized removal of AED 14. Station 16 may further contact safety agency 102 in response to the detected removal. For example, station 16 may send an advisory to safety agency 102 via communication unit 104A and network 106. In this manner, station 16 initiates direct communication between station 16 and safety agency 102. The communication may serve to request that emergency personnel be dispatched to the scene of the emergency. To that end, the communication may include location information, as well as other pertinent information.

Direct communication between station 16 and safety agency 102 may advantageously reduce the amount of time before delivery of early advanced care to the patient. Although described in terms of direct communication between station 16 and safety agency 102, system 100 may provide direct communication between the removed medical device (in this example AED 14) and safety agency 102. For example, upon detecting removal of AED 14 from station 16, AED 14 may sound an alarm located within AED 14 and initiate direct contact with safety agency 102 via communication unit 104B.

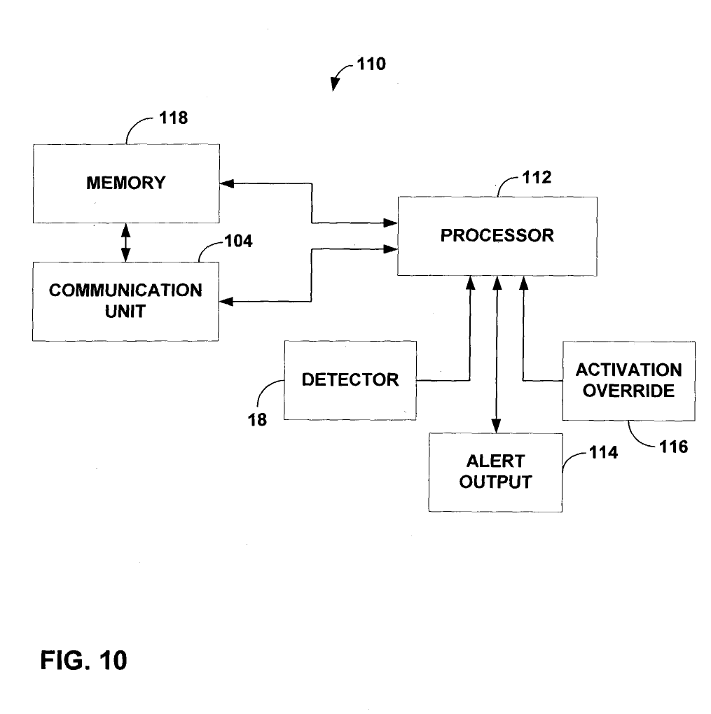

FIG. 10 is a block diagram illustrating an exemplary embodiment of communications circuitry 110 within station 16 for automatic initiation of direct contact between station 16 and a safety agency 102. As shown in FIG. 10, communications circuitry 110 includes a detector 18 that detects removal of AED 14 from station 16. For example, detector 18 may be an optical receiver that detects when AED 14 is removed from station 16 when an optical signal is no longer received.

Detector 18 causes alarm 12 to activate upon detecting removal of AED 14. More specifically, detector 18 may communicate to a processor 112 that removal of AED 14 was detected. Processor 112 conveys to an operator removing AED 14 from station 16, via an alert output 114 of the intent to contact safety agency 102. For example, processor 64 may convey to the operator that an advisory will be sent to safety agency 102. Alert output 114 may be a speaker, a display, or a combination thereof. Processor 112 may wait for a defined time interval after the alert to the operator before contacting safety agency 102.

The operator may choose to cancel the contact within the defined time interval via an activation override 116. The operator may choose to cancel the contact with safety agency 14, for example, when the event detected is a non-emergency situation. For example, an AED 14 may be removed from station 16 for routine maintenance, in which case there is no need to send an advisory to safety agency 102. Activation override 116 may, for example, be a button, switch, dial or other input medium that, when actuated by the operator, cancels the advisory. Alternatively, activation override 116 may take the form of an audible command from the operator.

When the operator does not cancel the contact within the defined time interval, processor 112 may access a memory 118 to generate an advisory. Memory 118 may include location information, such as a recorded message indicating the location of AED 14. Further, memory 118 may contain contact information of a prescribing physician, a serial number of the AED 14, and other pertinent information. Processor 112 may, for example, generate an advisory from a subset of the information stored in memory 118, and send the advisory to safety agency 102 via a communication unit 104.

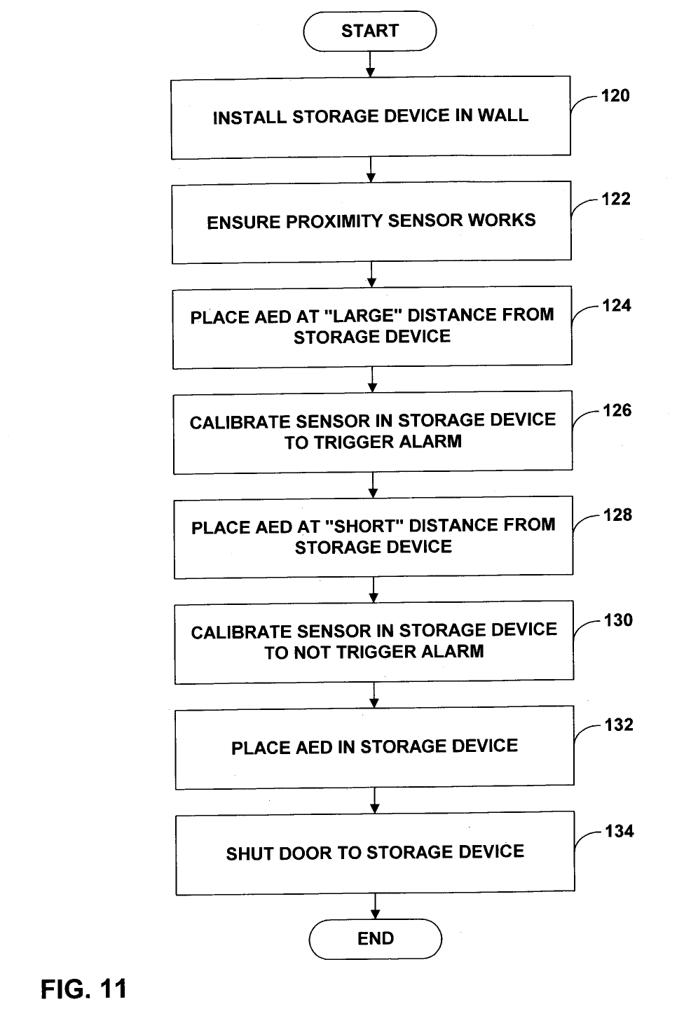

FIG. 11 is a flow diagram illustrating installation of station 16 and initialization of a detector 18 within station 16 for detecting removal of the medical device from station 16. Initially, station 16 is installed in a venue (120). Station 16 may, for example, be installed on a wall within the venue. Alternatively, station 16 may be coupled to a base and be free standing. In the case in which station 12 comprises a storage unit, such as storage unit 70, station 12 may be installed partially within the wall of the venue. The venue may include airports, health clubs, auditoriums and the like.

Next, detector 18 of station 16 is checked to ensure proper operation (122). Detector 18 may be checked, for example, by powering-up a relevant detection circuit. The medical device to be stored within/on station 16 is placed at a distance away from station 16 that it is desired for alarm 12 to activate (124). For example, for a wireless detector that uses radio frequency (RF) communication, the distance from station 16 that may activate alarm 12 may be 10-20 feet. Detector 18 is calibrated to trigger alarm 12 at the current distance (126). Calibration of detector 18 may, for example, include adjusting a sensitivity knob, such as knob 60 of FIG. 5.

The medical device is moved to a distance closer to station 16 and detector 18 is calibrated to not activate alarm 12 (128, 130). The closer distance, for example, may be only a few feet. Alternatively, the medical device may be placed within or on station 16 when the medical device is moved closer. However, if this is not the case, the medical device is then placed within or on station 16 after calibration of detector 18 (132).

If station 16 is a storage unit 70, a door section 80 of station 16 is closed (134). At this point installation and initialization are complete. Station 16 is now able to detect when the medical device is removed from station 16, e.g., when the medical device is removed outside of the sensing range.

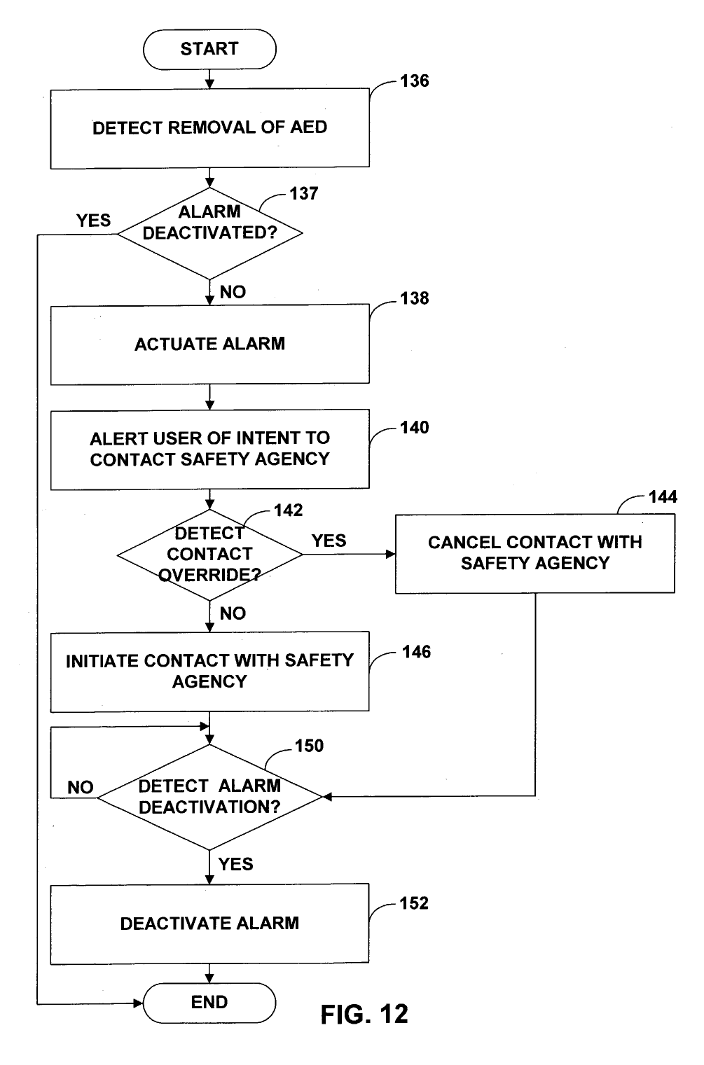

FIG. 12 is a block diagram illustrating operation of station 16 upon detecting removal of AED 14 from station 16. Initially, detector 18 detects removal of AED 14 from station 16 (136). For example, an optical detector may no longer receive an optical signal from AED 14 when AED 14 is moved beyond a detection range. Station 16 next determines whether alarm 12 is deactivated (137). When alarm 12 is deactivated, alarm 12 does not sound upon removal of AED 14 from station 16. Further, station 16 does not initiate contact between station 16 and safety agency 102. Alarm 12 may be deactivated, for example, when authorized personnel are performing routing maintenance to the medical device.

When alarm 12 is not deactivated, alarm 12 is sounded (138). A signal may be sent to actuate alarm 12 in response to detecting removal of AED 14. Alarm 12 may, for example, sound a buzzer or a siren as well as initiate a strobe light. Further, station 16 alerts the operator that removed AED 14 of the intent to contact safety agency 102 (140). The alert to the operator may be displayed on a display or may be prompted via a speaker located on station 16. The alert may indicate, for example, that an advisory will be sent to safety agency 102 in a defined amount of time unless the operator indicates otherwise. Station 16 may monitor for an override command to be input by the operator during the defined amount of time (142). In locations where automatic direct communication with the safety agency is not permitted by law, an override button, switch, dial, or other input medium may be present to allow the operator input an override command to cancel the advisory. When station 16 detects an override command from the operator, station 16 cancels the advisory to safety agency 102 (144).

When station 16 does not detect an override command from the operator, station 16 initiates communication with safety agency 102 (146). Processor 112 may, for example, retrieve information stored in memory 118, such as location information 66, contact information of a prescribing physician, and a serial number of the medical device and generate the advisory with a subset of the information. Station 16 may send the advisory to safety agency 102 via communication unit 104A. Alternatively, a voice channel may be opened between station 16 and safety agency 102.

Alarm 12 determines whether an alarm deactivation has occurred and deactivates alarm 12 upon receiving the alarm deactivation (150, 152). The alarm deactivation may include actuation of an alarm deactivation button, turning of a deactivation switch, or the like. For example, a person may use a key to turn the deactivation switch to deactivate alarm 12. The key deactivation switch allows deactivation of alarm 12 by authorized personnel only. Alternatively, alarm deactivation may occur upon expiration of an alarm deactivation timer.

As mentioned above, a medical device supported by the station 16 may instead provide the removal detection techniques provided by station 16. For example, AED 14 may detect removal by sensing the presence of station 16 and activate an alarm within or on AED 14 in response to the detected removal. Further, AED 14 may initiate direct contact with safety agency 102.

Various embodiments of the invention have been described. These embodiments are illustrative of the practice of the invention. Various modifications may be made without departing from the scope of the claims. The techniques of the invention may be applied to other medical devices that may be housed within a storage unit or otherwise supported by a station. These and other embodiments are within the scope of the following claims.

1. A system comprising:
a station to support a medical device;
a detector to detect removal of the medical device from the station; and
an alarm that is activated in response to the detected removal of the medical device.

2. The system of claim 1, in which the alarm includes an audible alarm.

3. The system of claim 2, in which the audible alarm includes one of a siren and a buzzer.

4. The system of claim 1, in which the alarm includes a visual alarm.

5. The system of claim 4, in which the visual alarm includes a strobe light.

6. The system of claim 1, in which the detector includes a proximity sensor.

7. The system of claim 6, in which the proximity sensor includes a mechanical switch that is actuated upon removal of the medical device from the station.

8. The system of claim 7, in which the mechanical switch includes a micro switch on a bottom portion of the station.

9. The system of claim 7, in which the mechanical switch includes one of a pin switch and a plate switch.

10. The system of claim 7, in which the mechanical switch includes a suspended mechanical switch.

11. The system of claim 6, in which the proximity sensor includes a capacitive sensor that senses a change in capacitance upon removal of the medical device from the station.

12. The system of claim 6, in which the proximity sensor includes a wireless sensor.

13. The system of claim 12, in which the detector detects removal of the medical device from the station when the wireless sensor no longer receives a signal from the medical device.

14. The system of claim 6, in which the proximity sensor includes an optical sensor.

15. The system of claim 14, in which the detector detects removal of the medical device from the station when the optical sensor no longer receives an optical signal from the medical device.

16. The system of claim 6, in which the proximity sensor includes a receptacle plug coupled to a wire.

17. The system of claim 16, in which the detector detects removal of the medical device from the station when the receptacle plug is unplugged from the medical device.

18. The system of claim 16, in which the receptacle plug is coupled to the station and the wire is coupled to the medical device.

19. The system of claim 6, in which the proximity sensor includes an emitter-detector circuit.

20. The system of claim 1, in which the station includes an storage unit comprising a plurality of wall sections, a top section, a bottom section, and a door section, in which the wall sections, the top section, the bottom section, and the door section define an interior compartment to house the medical device.

21. The system of claim 20, in which the station includes a mount within the interior compartment.

22. The system of claim 1, further comprising a deactivation switch to deactivate the alarm.

23. The system of claim 22, in which the deactivation switch is responsive to a timer and deactivates the alarm when the timer exceeds a threshold time.

24. The system of claim 22, in which the deactivation switch is responsive to a key.

25. The system of claim 1, further comprising a communication unit to contact a safety agency in response to detecting removal of the medical device from the station.

26. The system of claim 25, in which the communication unit sends an advisory to the safety agency in response to detecting removal of the medical device from the station.

27. The system of claim 26, in which the advisory includes at least one of location information, contact information of a prescribing physician, and a serial number of the medical device.

28. The system of claim 25, in which the communication unit is located within the medical device.

29. The system of claim 25, in which the communication unit is coupled to the station.

30. The system of claim 25, in which the communication unit includes at least one of a mobile phone, a wireless local area network (WLAN) card, a infrared (IR) card, a network card, and a modem.

31. The system of claim 1, in which the medical device is an automated external defibrillator (AED)

32. A method comprising:
detecting removal of a medical device from a station that supports the medical device; and
activating an alarm in responsive to the detected removal of the medical device from the station.

33. The method of claim 32, further comprising deactivating the alarm.

34. The method of claim 33, in which deactivating the alarm includes deactivating the alarm after a defined time interval.

35. The method of claim 34, in which deactivating the alarm includes deactivating the alarm permanently.

36. The method of claim 34, in which deactivating the alarm includes deactivating the alarm via one of a switch, a button, a key, and a dial.

37. The method of claim 32, further comprising contacting a safety agency in response to the detected removal.

38. The method of claim 37, in which contacting the safety agency includes sending an advisory to the safety agency.

39. The method of claim 38, in which the advisory is sent from the medical device.

40. The method of claim 38, in which the advisory is sent from the station.

41. The method of claim 38, in which the advisory includes at least one of location information, contact information of a prescribing physician, and a serial number of the medical device.

42. The method of claim 38, in which the advisory includes a recorded message.

43. The method of claim 32, in which the station includes a storage unit to house the medical device and the storage unit is opened to access the medical device.

44. The method of claim 32, in which the medical device includes an automated external defibrillator (AED).

45. A system comprising:
a station to support a medical device;
means for detecting removal of the medical device from the station; and
means for alerting people in the vicinity that the medical device has been removed.

46. The system of claim 45, further comprising means for deactivating the alerting means.

47. The system of claim 45, further comprising means for contacting a safety agency in response to detecting removal of the medical device.

48. The system of claim 45, in which the station includes a storage unit.

49. The system of claim 45, in which the detecting means include a proximity sensor.

50. The system of claim 45, in which the alerting means include an alarm.
